(19) United States
(12) Patent Application Publication
(10) Pub. No.: US 2009/0036339 A1
(43) Pub. Date: Feb. 5, 2009

(54) MILD, FOAMING CLEANSING COMPOSITION

(76) Inventors: ANNE SANS, Amfreville Suriton (FR); MARILYNE CANDOLIVES, Plainsboro, NJ (US); SYLVIE HAUCHECORNE, La Neuville Chant D'Oisel (FR)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/056,616

(22) Filed: Mar. 27, 2008

(30) Foreign Application Priority Data
Mar. 30, 2007 (EP) 07290379.2

(51) Int. Cl. A61K 8/04; A61Q 5/02; A61Q 5/12; A61Q 19/10

(52) U.S. Cl. 510/119; 510/158

(57) ABSTRACT

A mild cleansing composition that provides good foaming, yet is free of irritating anionic surfactants, is provided. The composition comprises a combination of amphoteric and nonionic surfactants.

The present invention relates to a mild, foaming cleansing composition that is advantageously free of anionic surfactants.

### BACKGROUND OF THE INVENTION

Cleansing compositions generally contain anionic surfactants as a primary ingredient. Anionic surfactants typically provide the foaming in such compositions. Foaming, in turn, is directly related to perceived cleansing efficacy. The greater the volume of foam produced and the greater the stability of the foam, the more efficient the perceived cleansing action.

Unfortunately, anionic surfactants are also irritating to the skin and eyes. Accordingly, cleansing compositions, such as bath gels, shower gels, shampoos and the like that do not rely on anionic surfactants would be highly desirable. This is particularly true in the case of baby products, products for sensitive skin, and “natural” products.

Similarly, products that are free of polyethylene glycol (PEG) and ethoxylates are also desirable. Certain literature has indicated that PEG and PEG derivatives are suspected to contain impurities due to their production processes, like dioxan or ethylene oxide, which are known for their carcinogenic effects. In addition, they may also facilitate penetration of raw materials that may be irritants into the skin.

Applicants have discovered that a combination of amphoteric surfactants and nonionic surfactants without any anionic surfactants can provide stable cleansing compositions having both mildness and good foaming. Such compositions may be clear or opaque. In a further embodiment, such compositions are also free of PEG and ethoxylates, providing excellent cleansing products for use on sensitive skin.

### SUMMARY OF THE INVENTION

The invention provides a cleansing composition comprising: 1), in particular from 0.1 to 40 weight % of, at least one amphoteric surfactant, and 2), in particular from 0.1 to 40 weight % of, at least one nonionic surfactant, wherein said composition is free from anionic surfactants, and the total amount of surfactants in said composition is up to 70 weight %. These compositions preferably have a TEP score of at least 2, and/or a foam volume of at least 500 mL.

The invention further provides such a composition that is also free from PEG and ethoxylated derivatives.

### DETAILED DESCRIPTION OF THE INVENTION

All weight percents are based on the total weight of the composition.

The composition comprises up to, i.e. not more than about 70 weight % of surfactants. The surfactants are a combination of one or more amphoteric surfactants and one or more nonionic surfactants. The composition is free of anionic surfactants and in one embodiment, free of PEG and ethoxylated derivatives. That is, the composition contains less than an effective amount of anionic surfactants, preferably no anionic surfactants.

In one embodiment, the ratio of amphoteric surfactant to nonionic surfactant is in the range of about 25:1 to about 1:25. Preferably, the ratio of amphoteric surfactant to nonionic surfactant is in the range of about 9:1 to about 1:2, preferably about 2:1.

### Amphoteric Surfactants

The amount of amphoteric surfactant in the composition preferably ranges from about 0.1 to about 40, preferably from about 5 to about 30 weight percent of the composition.

The term “amphoteric surfactant,” as used herein, is also intended to encompass zwitterionic surfactants, which are well known to formulators skilled in the art as a subset of amphoteric surfactants.

A wide variety of amphoteric surfactants can be used. Particularly useful are those which are broadly described as derivatives of aliphatic secondary and tertiary amines, preferably wherein the nitrogen is in a cationic state, in which the aliphatic radicals can be straight or branched chain and wherein one of the radicals contains an ionizable water solubilizing group, e.g., carboxy, sulfonate, sulfate, phosphate, or phosphonate.

Nonlimiting examples of amphoteric surfactants are disclosed in McCutcheon's, Detergents and Emulsifiers, North American edition (1986), published by Allured Publishing Corporation; and McCutcheon's, Functional Materials, North American Edition (1992); both of which are incorporated by reference herein in their entirety.

Nonlimiting examples of amphoteric or zwitterionic surfactants are those selected from the group consisting of betaines, sultaines, hydroxysultaines, alkyliminoacetates, iminodialkanoates, aminoalkanoates, and mixtures thereof.

Examples of betaines include the higher alkyl betaines, such as coco dimethyl carboxymethyl betaine, lauryl dimethyl carboxymethyl betaine, lauryl dimethyl alphacarboxyethyl betaine, cetyl dimethyl carboxymethyl betaine, cetyl dimethyl betaine (e.g. available as Lonzaine 16SP from Lonza Corp.), lauryl bis-(2-hydroxyethyl)carboxymethyl betaine, oleyl dimethyl gamma-carboxypropyl betaine, lauryl bis-(2-hydroxypropyl)alpha-carboxyethyl betaine, coco dimethyl sulfopropyl betaine, lauryl dimethyl sulfoethyl betaine, lauryl bis-(2-hydroxyethyl)sulfopropyl betaine, amidobetaines and amidosulfobetaines (wherein the RCONH(CH₂)₃ radical is attached to the nitrogen atom of the betaine and wherein R may have the meaning of R¹ as defined below), oleyl betaine (e.g. available as amphoteric Velvetex OLB-50 from Henkel), and cocamidopropyl betaine (e.g. available as Velvetex BK-35 and BA-35 from Henkel).

Examples of sultaines and hydroxysultaines include materials such as cocamidopropyl hydroxysultaine (e.g. available as Mirataine CBS from Rhone-Poulenc).

Preferred for use herein are amphoteric surfactants having the following structure:

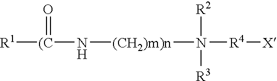

wherein R₁ is (in particular unsubstituted) saturated or unsaturated, straight or branched chain alkyl having from about 9 to about 22 carbon atoms. Preferred R₁ has from about 11 to about 18 carbon atoms; more preferably from about 12 to about 18 carbon atoms; more preferably still from about 14 to about 18 carbon atoms; m is an integer from 1 to about 3, more preferably from about 2 to about 3, and more preferably about 3; n is either 0 or 1, preferably 1; R₂ and R₃ are independently selected from the group consisting of alkyl having from 1 to about 3 carbon atoms, unsubstituted or mono-substituted with hydroxyl, preferred R₂ and R₃ are CH₃; X is selected from the group consisting of CO₂, SO₃ and SO₄; R₄ is selected from the group consisting of saturated or unsaturated, straight or branched chain alkyl, unsubstituted or monosubstituted with hydroxyl, having from 1 to about 5 carbon atoms. When X is CO₂, R₄ preferably has 1 or 3 carbon atoms, more preferably 1 carbon atom. When X is SO₃ or SO₄, R₄ preferably has from about 2 to about 4 carbon atoms, more preferably 3 carbon atoms.

Examples of amphoteric surfactants of the present invention include the following compounds: Cetyl dimethyl betaine (this material also has the CTFA designation cetyl betaine)

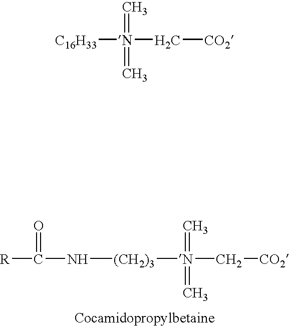

wherein R has from about 9 to 18, e.g. in particular from about 9 to about 13, or from 14 to 18 carbon atoms

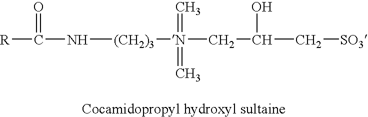

wherein R has from about 9 to 18, e.g. in particular from about 9 to about 13, or from 14 to 18, carbon atoms.

Examples of other useful amphoteric surfactants are alkyliminoacetates, and iminodialkanoates and aminoalkanoates of the formulas RN[CH₂)ₘCO₂M]₂ and RNH(CH₂)ₘCO₂M wherein m is from 1 to 4, R is a C₈-C₂₂ alkyl or alkenyl, and M is H, alkali metal, alkaline earth metal ammonium, or alkanolammonium. Also included are imidazolinium and ammonium derivatives. Specific examples of suitable amphoteric surfactants include sodium 3-dodecyl-aminopropionate, sodium 3-dodecylamino-propane sulfonate, N-higher alkyl aspartic acids such as those produced according to the teaching of U.S. Pat. No. 2,438,091 which is incorporated herein by reference in its entirety; and the products sold under the trade name “Miranol” and described in U.S. Pat. No. 2,528,378, which is incorporated herein by reference in its entirety. Other examples of useful amphoterics include amphoteric phosphates, such as coamidopropyl PG-dimonium chloride phosphate (e.g. commercially available as Monaquat PTC, from Mona Corp.). Also useful are amphoacetates such as disodium lauroamphodiacetate, sodium lauroamphoacetate, and mixtures thereof.

Preferred amphoteric surfactants are selected from the group consisting of disodium lauroamphodiacetate, sodium lauroamphoacetate, cetyl dimethyl betaine, cocoamidopropyl betaine, cocoamidopropyl hydroxyl sultaine, and mixtures thereof.

### Nonionic Surfactants

The amount of nonionic surfactant in the composition preferably ranges from about 0.1 to about 40 weight percent of the composition. More preferably, the amount of nonionic surfactant ranges from about 1 to about 20 weight percent of the composition.

Nonlimiting examples of nonionic surfactants are disclosed in McCutcheon's, Detergents and Emulsifiers, North American edition (1986), published by allured Publishing Corporation; and McCutcheon's, Functional Materials, North American Edition (1992); both of which are incorporated by reference herein in their entirety.

Nonionic surfactants useful herein include those selected from the group consisting of alkyl glucosides, alkyl polyglucosides, polyhydroxy fatty acid amides, sucrose esters, amine oxides, and mixtures thereof.

Alkyl glucosides and alkyl polyglucosides are useful herein, and can be broadly defined as condensation products of long chain alcohols, e.g., C8-C30 alcohols, with sugars or starches or sugar or starch polymers, i.e., glycosides or polyglycosides. These compounds can be represented by the formula (S)ₙ—O—R wherein S is a sugar moiety such as glucose, fructose, mannose, and galactose; n is an integer of from about 1 to about 1000, and R is a C8-C30 alkyl group. Examples of long chain alcohols from which the alkyl group can be derived include decyl alcohol, cetyl alcohol, stearyl alcohol, lauryl alcohol, myristyl alcohol, oleyl alcohol, and the like. Preferred examples of these surfactants include those wherein S is a glucose moiety, R is a C8-C20 alkyl group and n is an integer of from about 1 to about 9. Commercially available examples of these surfactants include decyl polyglucoside (e.g. available as APG 325 CS from Henkel) and lauryl polyglucoside (e.g. available as APG 600CS and 625 CS from Henkel). Also useful are sucrose ester surfactants such as sucrose cocoate and sucrose laurate.

Other useful nonionic surfactants include polyhydroxy fatty acid amide surfactants, more specific examples of which include glucosamides, corresponding to the formula:

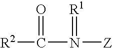

wherein: R¹ is H, C₁-C₄ alkyl, 2-hydroxyethyl, 2-hydroxy-propyl, preferably C₁-C₄ alkyl, more preferably methyl or ethyl, most preferably methyl; R² is C₅-C₃₁ alkyl or alkenyl, preferably C₇-C₁₉ alkyl or alkenyl, more preferably C₉-C₁₇ alkyl or alkenyl, most preferably C₁₁-C₁₅ alkyl or alkenyl; and Z is a polhydroxyhydrocarbyl moiety having a linear hydrocarbyl chain with a least 3 hydroxyls directly connected to the chain, thereof. Z preferably is a sugar moiety selected from the group consisting of glucose, fructose, maltose, lactose, galactose, mannose, xylose, and mixtures thereof. An especially preferred surfactant corresponding to the above structure is coconut alkyl N-methyl glucoside amide (i.e., wherein the R²CO— moiety is derived from coconut oil fatty acids). Processes for making compositions containing polyhydroxy fatty acid amides are disclosed, for example, in G.B. Patent Specification 809,060; U.S. Pat. No. 2,965,576, U.S. Pat. No. 2,703,798, and U.S. Pat. No. 1,985,424, each of which are incorporated herein by reference in their entirety.

Other examples of nonionic surfactants include amine oxides. Amine oxides correspond to the general formula R₁, R₂, R₃ N—O, wherein e.g. R₁ contains an alkyl, alkenyl or monohydroxy alkyl radical of from about 8 to about 18 carbon atoms, and R₂ and R₃ contain from about 1 to about 3 carbon atoms and from 0 to about 1 hydroxy group, e.g., methyl, ethyl, propyl, hydroxyethyl, or hydroxypropyl radicals. Examples of amine oxides suitable for use in this invention include dimethyl-dodecylamine oxide, oleyidi(2-hydroxyethyl)amine oxide, dimethyloctylamine oxide, dimethyl-decylamine oxide, dimethyl-tetradecylamine oxide, 3,6,9-trioxaheptadecyldiethylamine oxide, di(2-hydroxyethyl)-tetradecylamine oxide, 2-dodecoxyethyldimethylamine oxide, 3-dodecoxy-2-hydroxypropyldi(3-hydroxypropyl)amine oxide, dimethylhexadecylamine oxide.

Nonlimiting examples of preferred nonionic surfactants for use herein are those selected form the group consisting of C8-C14 glucose amides, C8-C14 alkyl polyglucosides such as cocoglucoside, sucrose cocoate, sucrose laurate, lauramine oxide, cocoamine oxide, lauryl glucoside, decyl glucoside, and mixtures thereof.

In one embodiment, the composition is also free of PEG and ethoxylated derivatives. Such materials are typically added to cleansing compositions for the purpose of thickening, solubilizing, or adjusting the viscosity. The present composition, however, in a preferred embodiment contains less than an effective amount of PEG and ethoxylated derivatives, preferably no PEG or ethoxylated derivatives.

### Humectant

The composition may also optionally contain at least one humectant. The humectant may be selected from a variety of known compounds. Examples of suitable humectants nonexclusively include: 1) water soluble liquid polyols selected from the group comprising glycerine, propylene glycol, hexylene glycol, butylene glycol, dipropylene glycol, and mixtures thereof, 2) urea; and 3) mixtures thereof, with glycerine being the preferred humectant.

The amount of humectant in the composition may range from about 0.1 to about 50 weight percent. Preferably, the amount of humectant ranges from about 5 to about 15.

### Thickener

The composition may optionally contain at least one thickener. The thickener may be selected from a variety of known compounds. Examples of suitable thickeners nonexclusively include fatty acids containing from about 16 to about 22 carbon atoms; hydroxyalkyl cellulose; alkyl cellulose; hydroxyalkyl alkyl cellulose; xanthan gum, acrylates/C10-30 alkyl acrylate crosspolymer, carbomer, silicas, salts such as sodium chloride, and mixtures thereof.

The amount of thickener in the composition may range from about 0.01 to about 20 weight percent. Preferably, the amount of thickener ranges from about 0.1 to about 10 weight percent.

The composition may also contain various preservatives, conditioning agents, fragrances, etc., as known in the art, which are preferably also free of ethoxylated derivatives and PEG.

### Clarity

The composition of the invention may be clear or opaque. In one embodiment, the composition is clear and exhibits a light transmittance of at least about 50%, preferably at least about 90%, most preferably at least about 98%, as measured using a UV spectrophotometer, for example a Model DU Beckman UV spectrophotometer, at a wavelength of 800 nm and utilizing a 1 cm cell.

### Foaming

Despite the absence of anionic surfactants in the composition, it demonstrates good foaming. In particular, the composition provides a foam volume of at least 500 mL, preferably at least about 700 mL, more preferably at least about 800 mL. Foam volume is measured using a SITA Foam Tester using the body wash setting as follows. A stirrer is used to create foam in a temperature-controlled glass vessel containing a pre-determined volume of the composition, which is stirred for a pre-determined time. The foam volume is measured using sensors across the whole surface area. The foam volume measured over time without stirring (foam stability) can also be determined.

### Mildness—TEP

The composition is also advantageously mild. In particular, it possesses a relatively high TEP value, i.e., a passing TEP score as determined in accordance with the TEP Test as set forth herein. In particular, the composition has a TEP score of at least 1.6, preferably at least 2, and more preferably at least 2.2.

Trans-Epithelial Permeability Test (“TEP Test”): Irritation to the eyes expected for a given formulation is measured in accordance with the Invittox Protocol Number 86, the “Trans-epithelial Permeability (TEP) Assay” as set forth in Invittox Protocol Number 86 (May 1994). In general, the ocular irritation potential of a product can be evaluated by determining its effect on the permeability of a cell layer, as assessed by the leakage of fluorescein through the layer. Monolayers of Madin-Darby canine kidney (MDCK) cells are grown to confluence on microporous inserts in a 24-well plate containing medium or assay buffer in the lower wells. The irritation potential of a product is evaluated by measuring the damage to the permeability barrier in the cell monolayer following a 15 minute exposure to dilutions of the product. Barrier damage is assessed by the amount of sodium fluorescein that has leaked through to the lower well after 30 minutes, as determined spectrophotometrically. The fluorescein leakage is plotted against the concentration of test material to determine the EC50 (the concentration of test material that causes 50% of maximum dye leakage, i.e., 50% damage to the permeability barrier). Higher scores are indicative of milder formulas.

Exposure of a layer of MDCK cells grown on a microporous membrane to a test sample is a model for the first event that occurs when an irritant comes in contact with the eye. In vivo, the outermost layers of the corneal epithelium form a selectively permeable barrier due to the presence of tight junctions between cells. On exposure to an irritant, the tight junctions separate, thereby removing the permeability barrier. Fluid is imbibed to the underlying layers of epithelium and to the stroma, causing the collagen lamellae to separate, resulting in opacity. The TEP assay measures the effect of an irritant on the breakdown of tight junctions between cells in a layer of MDCK cells grown on a microporous insert. Damage is evaluated spectrophotometrically, by measuring the amount of marker dye (sodium fluorescein) that leaks through the cell layer and microporous membrane to the lower well.

The composition of the present invention may be used in personal care products such as shampoos, washes, baths, rinse off conditioners, gels, lotions, creams, and the like. The composition may also be used in conjunction with cleansing implements such as wipes, poufs, sponges, cloths, and the like, or may be impregnated therein. The composition may also be combined with such implements for convenient sale and use in the form of a kit

The following non-limiting examples further illustrate the invention.

### EXAMPLES

The following compositions according to the invention, Examples 1-7, were prepared. They were tested for TEP and foam volume. The results are shown in Table 1 below.

In addition, Comparative Examples A-D were also tested for TEP and foam volume. Comparative Examples A, B, and C were Baby Love Shampoo, Washbalsam, and Pflegebad, all commercially available from Dm-drogerie Markt. Comparative Example D was HIPP Pflegebad, which is commercially available from Hipp GmbH & Co Vertrieb KG. The results are shown in Table 2.

TABLE 1

| | Example 1 | Example 2 | Example 3 | Example 4 | Example 5 | Example 6 | |
|---|---|---|---|---|---|---|---|
| Ingredient | Weight % | Weight % | Weight % | Weight % | Weight % | Weight % | Example 7 |
| Cocamidopropyl Betaine | 13.58 | 5.90 | 13.51 | 11.00 | 6.00 | 7.89 | 8.75 |
| Sodium Lauroamphoacetate | 10.58 | 23.80 | | | | 10.63 | 12.96 |
| Cocoglucoside | 3.11 | 2.20 | 10.00 | 12.00 | 16.00 | 9.25 | 7.00 |
| Polyquaternium-7 | 1.20 | 1.20 | | | | | |
| Coco-glucoside Glyceryl Oleate | | | 1.00 | 1.00 | 1.00 | 1.00 | 1.00 |
| Glycerin | 5.00 | 5.00 | | | | | |
| Olea Europaea | 0.01 | 0.01 | | | | | |
| Helianthus Annuus, Tocopherol | 0.01 | 0.01 | | | | | |
| Sodium Cocoyl amino acids/ | 0.01 | 0.01 | | | | | |
| sarcosine/potassium aspartate/ | | | | | | | |
| magnesium aspartate/Aqua | | | | | | | |
| Tocopheryl acetate | 0.09 | 0.09 | | | | | |
| Aloe Barbadensis Leaf Extract/ | 0.01 | 0.01 | | | | | |
| glycerin/phenoxyethanol/Aqua | | | | | | | |
| Chamomilla Recutita Flower Extract/ | 0.01 | 0.01 | | | | | |
| Glycerin/Phenoxyethanol | | | | | | | |
| Acrylates/C10-30 alkyl Acrylate | 0.60 | 0.50 | | | | | |
| Crosspolymer | | | | | | | |
| XANTHAN GUM | | | 0.50 | 0.63 | 0.63 | 0.75 | |
| Acrylate Copolymer | | | | | | | |
| Hydroxypropyl Strach Phosphate | 1.50 | 1.50 | | | | | |
| Parfum | 0.40 | 0.40 | 0.10 | 0.10 | 0.10 | 0.10 | 0.10 |
| Sodium Benzoate | 0.50 | 0.50 | 0.50 | 0.50 | 0.50 | 0.50 | 0.50 |
| p-Anisic acid | | | 0.20 | 0.20 | 0.20 | 0.20 | 0.20 |
| Phenoxyethanol | | | 0.70 | 0.70 | 0.70 | 0.70 | 0.70 |
| Styrene/Acrylates copolymer | 0.80 | 0.80 | | | | | |
| Glycol distearate | | | | | | | |
| Disodium EDTA | 0.10 | 0.10 | | | | | |
| Sodium Hydroxyde | 0.04 | 0.04 | | 0.18 | 0.20 | 0.23 | 0.20 |
| Citric Acid | 0.60 | 1.40 | 0.04 | 0.04 | 0.17 | 0.60 | 0.71 |
| TEP Score | 2.42 +/− 0.13 | 2.20 +/− 0.55 | 2.54 +/− 0.49 | 2.80 +/− 0.44 | 3.25 +/− 0.25 | | 2.25 +/− 0.52 |
| Foam Volume | 797 mL | 577 mL | 868 mL | 820 mL | 728 mL | 848 mL | — |

TABLE 2

| Comparative | Comparative | Comparative | Comparative |
|---|---|---|---|
| Example A | Example B | Example C | Example D |
| AQUA | AQUA | AQUA | AQUA |
| LAURYL GLUCOSIDE | LAURYL GLUCOSIDE | LAURYL GLUCOSIDE | SORBITOL |
| COCAMIDOPROPYL | COCAMIDOPROPYL | COCAMIDOPROPYL BETAINE | COCAMIDOPROPYL |
| BETAINE | BETAINE | COCO GLUCOSIDE | HYDROXYSULTAINE |
| COCO GLUCOSIDE | SODIUM LAURYL GLUCOSE | GLYCERYL OLEATE | COCAMIDOPROPYL BETAINE |
| GLYCERYL OLEATE | CARBOXYLATE | SODIUM CITRATE | LAURYL GLUCOSIDE |
| PANTHENOL | GLYCERIN | PANTHENOL | SODIUM CHLORIDE |
| TRITICUM VULGARE | ACRYLATES COPOLYMER | GLYCERYL CAPRYLATE | PRUNUS AMYGDALUS DULCIS |
| CHAMOMILLA RECUTITA | COCO GLUCOSIDE | CHAMOMILLA RECUTITA | SODIUM CARBOXYMETHYL |
| BISABOLOL | GLYCOL DISTEARATE | BISABOLOL | BETAGLUCAN |
| POTASSIUM SORBATE | GLYCERYL OLEATE | CITRIC ACID | COCO GLUCOSIDE |
| SODIUM BENZOATE | GLYCOL STEARATE | PARFUM | GLYCERYL OLEATE |
| SODIUM METHYLPARABEN | TRITICUM VULGARE | | GLYCERIN |
| SODIUM PROPYLPARABEN | PANTHENOL | | STYRENE/ACRYLATES COPOLYMER |
| CITRIC ACID | TOCOPHERYL ACETATE | | CITRIC ACID |
| PARFUM | DICAPRYLYL ETHER | | PARFUM |
| | LAURYL ALCOHOL | | |
| | SODIUM HYDROXIDE | | |
| | POTASSIUM SORBATE | | |
| | SODIUM METHYLPARABEN | | |
| | ALCOHOL | | |
| | PHENETHYL ALCOHOL | | |
| | MYRISTYL LACTATE | | |
| | ALEURITIC ACID | | |
| | PARFUM | | |
| | | | TEP Score = 0.82 +/− 0.27 |
| Foam Volume = 328 L | Foam Volume = 475 mL | Foam Volume = 366 mL | Foam Volume = 870 mL |

What is claimed is:

1. A cleansing composition comprising 1) at least one amphoteric surfactant, and 2) at least one nonionic surfactant, wherein said composition is free from anionic surfactants and the total amount of surfactants in said composition is up to 70 weight %.

2. The composition according to claim 1 which comprises: 1) 0.1 to 40 weight % of at least one amphoteric surfactant, and 2) 0.1 to 40 weight % of at least one nonionic surfactant, wherein said composition is free from anionic surfactants, and the total amount of surfactants in said composition is up to 70 weight %.

3. The composition according to claim 1 or 2 which has a TEP score of at least 1.6, preferably of at least 2, and/or a foam volume of at least 500 ml.

4. A cleansing composition, in particular according to any of the preceding claims, comprising: 1) 0.1 to 40 weight % of at least one amphoteric surfactant, and 2) 0.1 to 40 weight % of at least one nonionic surfactant, wherein said composition is free from anionic surfactants, has a TEP score of at least 2, and a foam volume of at least 500 mL, and the total amount of surfactants in said composition is up to 70 weight %.

5. The composition according to any of the preceding claims wherein the weight ratio of amphoteric surfactant to nonionic surfactant is in the range of 25:1 to 1:25.

6. The composition according to any of the preceding claims wherein the weight ratio of amphoteric surfactant to nonionic surfactant is in the range of 9:1 to 1:2.

7. The composition according to any of the preceding claims which is also free from polyethylene glycol.

8. The composition according to any of the preceding claims which is also free from ethoxylated derivatives.

9. The composition according to any of claims 1 to 6 which is also free from polyethylene glycol and ethoxylated derivatives.

10. The composition according to any of the preceding claims that is clear.

11. The composition according to any of claims 1 to 9 that is opaque.

12. The composition according to any of the preceding claims further comprising at least one humectant, thickener, and/or conditioner, preferably free of polyethylene glycol and/or ethoxylated derivatives.

13. A bath gel, shower gel, facial wash, rinse off conditioner, wipe, or shampoo comprising the composition according to any of the preceding claims.

14. Use of a composition according to any of claims 1 to 12 as a bath gel, shower gel, facial wash, rinse off conditioner, wipe, or shampoo or as a part thereof.
